(19) United States
(12) Patent Application Publication
(10) Pub. No.: US 2021/0065858 A1
(43) Pub. Date: Mar. 4, 2021

(54) GRAPHICAL METHOD FOR PRESENTING DIAGNOSTIC TEST PERFORMANCE RESULTS

(71) Applicant: KONINKLIJKE PHILIPS N.V., EINDHOVEN (NL)

(72) Inventor: JYH-YUN JOHN WANG, NEWTON, MA (US)

(21) Appl. No.: 16/857,844

(22) Filed: Apr. 24, 2020

Related U.S. Application Data
(60) Provisional application No. 62/895,205, filed on Sep. 3, 2019.

(51) Int. Cl. G16H 15/00; G06T 11/20; A61B 5/00

(52) U.S. Cl. CPC G16H 15/00; G06T 11/206; G06T 2200/24; A61B 5/7278; A61B 5/743

(57) ABSTRACT

A method for generating a graphical performance display, including: receiving performance measure data; generating a graphical rectangular plot, having a first side indicating a prevalence value, a second side indicating a sensitivity value, and third side indicating a specificity value, wherein the second and third sides are opposite one another and a fourth side is opposite the first side; drawing a prevalence line perpendicular to the first side between the first side and the fourth side based upon a prevalence value in the performance measure data; drawing a sensitivity line perpendicular to the second side between the second side and the prevalence line based upon a sensitivity value in the performance measure data; and drawing a specificity line perpendicular to the third side between the third side and the prevalence line based upon a sensitivity value in the performance measure data.

### TECHNICAL FIELD

Various exemplary embodiments disclosed herein relate generally to a graphical method for presenting diagnostic test performance results.

### BACKGROUND

Reporting and interpreting test performance results of detection and classification algorithms is required in many applications. For instance, numerous diagnostic tests are routinely performed clinically to: 1) screen for disease; 2) establish or rule-out a diagnosis; and 3) track and monitor disease progression and effectiveness of treatment. Thus, reporting and interpreting diagnostic test results is critical in supporting clinical decisions for the most effective patient management.

SUMMARY

Various embodiments relate to a summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

A method for generating a graphical performance display, including: receiving performance measure data; generating a graphical rectangular plot, having a first side indicating a prevalence value, a second side indicating a sensitivity value, and third side indicating a specificity value, wherein the second and third sides are opposite one another and a fourth side is opposite the first side; drawing a prevalence line perpendicular to the first side between the first side and the fourth side based upon a prevalence value in the performance measure data; drawing a sensitivity line perpendicular to the second side between the second side and the prevalence line based upon a sensitivity value in the performance measure data; and drawing a specificity line perpendicular to the third side between the third side and the prevalence line based upon a sensitivity value in the performance measure data.

Various embodiments are described, further comprising displaying the value of at least one of positive predictive value (PPV), negative predictive value (NPV), overall accuracy (ACC), positive likelihood ratio (LR+), negative likelihood ratio (LR−), positive pretest odds, negative pretest odds, positive posttest odds, and negative posttest odds

Various embodiments are described, further comprising calculating the displayed value based upon the received performance measure data.

Various embodiments are described, further including: displaying a normalized true positive value (TPn) in a first rectangular area bounded by the second side, fourth side, prevalence line, and sensitivity line, wherein the TPn value is related to the area of the first rectangular area;

displaying a normalized false negative value (FNn) in a second rectangular area bounded by the second side, first side, prevalence line, and sensitivity line, wherein the FNn value is related to the area of the second rectangular area; displaying a normalized false positive value (FPn) in a third rectangular area bounded by the third side, fourth side, prevalence line, and specificity line, wherein the FPn value is related to the area of the third rectangular area; and displaying a normalized true negative value (TNn) in a fourth rectangular area bounded by the third side, first side, prevalence line, and specificity line, wherein the TNn value is related to the area of the fourth rectangular area.

Various embodiments are described, further comprising calculating values for predictive value (PPV), negative predictive value (NPV), overall accuracy (ACC) using either the TPn, FNn, FPn, and TNn values or the areas for the first rectangular area, second rectangular area, third rectangular area, and fourth rectangular area.

Various embodiments are described, further including: receiving user input indicating a change in one of the prevalence, sensitivity, and specificity; displaying a change in the location of the prevalence line, sensitivity line, or the specificity line based upon the received user input; recalculating the displayed value; and displaying the recalculated value.

Various embodiments are described, wherein the user input includes selecting one of the prevalence line, sensitivity line, and specificity line and dragging it to a new position.

Various embodiments are described, wherein the user input includes inputting one of prevalence value, sensitivity value, and specificity value.

Various embodiments are described, further comprising generating and displaying a table of performance measure data and recalculated performance measure data.

Various embodiments are described, wherein a data scale for the second and third sides increase in opposite directions.

Further various embodiments relate to a non-transitory machine-readable storage medium encoded with instructions for generating a graphical performance display, including: instructions for receiving performance measure data; instructions for generating a graphical rectangular plot, having a first side indicating a prevalence value, a second side indicating a sensitivity value, and third side indicating a specificity value, wherein the second and third sides are opposite one another and a fourth side is opposite the first side; instructions for drawing a prevalence line perpendicular to the first side between the first side and the fourth side based upon a prevalence value in the performance measure data; instructions for drawing a sensitivity line perpendicular to the second side between the second side and the prevalence line based upon a sensitivity value in the performance measure data; and instructions for drawing a specificity line perpendicular to the third side between the third side and the prevalence line based upon a sensitivity value in the performance measure data.

Various embodiments are described, further comprising instructions for displaying the value of at least one of positive predictive value (PPV), negative predictive value (NPV), overall accuracy (ACC), positive likelihood ratio (LR+), negative likelihood ratio (LR−), positive pretest odds, negative pretest odds, positive posttest odds, and negative posttest odds

Various embodiments are described, further comprising instructions for calculating the displayed value based upon the received performance measure data.

Various embodiments are described, further including: instructions for displaying a normalized true positive value (TPn) in a first rectangular area bounded by the second side, fourth side, prevalence line, and sensitivity line, wherein the TPn value is related to the area of the first rectangular area; instructions for displaying a normalized false negative value (FNn) in a second rectangular area bounded by the second side, first side, prevalence line, and sensitivity line, wherein the FNn value is related to the area of the second rectangular area; instructions for displaying a normalized false positive value (FPn) in a third rectangular area bounded by the third side, fourth side, prevalence line, and specificity line, wherein the FPn value is related to the area of the third rectangular area; and instructions for displaying a normalized true negative value (TNn) in a fourth rectangular area bounded by the third side, first side, prevalence line, and specificity line, wherein the TNn value is related to the area of the fourth rectangular area.

Various embodiments are described, further including instructions for calculating values for predictive value (PPV), negative predictive value (NPV), overall accuracy (ACC) using either the TPn, FNn, FPn, and TNn values or the areas for the first rectangular area, second rectangular area, third rectangular area, and fourth rectangular area.

Various embodiments are described, further including: instructions for receiving user input indicating a change in one of the prevalence, sensitivity, and specificity; instructions for displaying a change in the location of the prevalence line, sensitivity line, or the specificity line based upon the received user input; instructions for recalculating the displayed value; and instructions for displaying the recalculated value.

Various embodiments are described, wherein the user input includes selecting one of the prevalence line, sensitivity line, or specificity line and dragging it to a new position.

Various embodiments are described, wherein the user input includes inputting one of prevalence value, sensitivity value, or specificity value.

Various embodiments are described, further comprising instructions for generating and displaying a table of performance measure data and recalculated performance measure data.

Various embodiments are described, wherein a data scale for the second and third sides increase in opposite directions.

### BRIEF DESCRIPTION OF THE DRAWINGS

In order to better understand various exemplary embodiments, reference is made to the accompanying drawings, wherein:

FIG. 1 illustrates an example of the graphical presentation for performance reporting;

FIGS. 2A and 2B illustrate how the graphical performance display is used to understand how changing the prevalence value affects various other performance measures;

FIGS. 3A-3D illustrate that three lines may be moved to increase TP (upper-left) area and/or decrease FP (upper-right) area to improve the PPV;

FIGS. 4A-4D illustrate ACC behavior for increased prevalence level for Se>Sp vs. Se<Sp;

FIGS. 5A and 5B illustrate the ACC paradox; and

FIG. 6 illustrates an exemplary hardware diagram for generating and displaying the graphical performance display illustrated in FIGS. 1-5.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

In order to better present the performance results of a diagnostic test, embodiments of a new graphical presentation for reporting the performance results of a diagnostic test is disclosed. By using line segments, ratio of line segments, areas, sum of areas, and ratio of areas, the graphical presentation is able to present in a single graph a total of 19 commonly used performance measures as defined below. The graphical presentation allows the relationships among the performance measures to be directly visualized and thus making the interpretation of the performance results much easier. Finally, with the aid of the graphical presentation, the equations used for calculating the performance measures can be visualized and generated easily from the graph and thus making the assessment of performance results using different parameter values more easily.

Reporting and interpreting test performance results of detection and classification algorithms is required in many applications. For instance, numerous diagnostic tests are routinely performed clinically to: 1) screen for disease; 2) establish or rule-out a diagnosis; and 3) track and monitor disease progression and effectiveness of treatment. Thus, reporting and interpreting diagnostic test results is critical in supporting clinical decisions for the most effective patient management.

The standard performance measures used in reporting the test results of a classifier are sensitivity (Se), specificity (Sp), overall accuracy (ACC), pretest likelihood (prevalence), and posttest likelihood including positive predictive value (PPV) and negative predictive value (NPV) and the corresponding positive posttest odds and negative posttest odds. The definitions and formulas for these statistical performance measures are summarized in Table 1. As shown in the table, the efficacy of a test is entirely captured by the following four basic measurements: true positive (TP), false negative (FN), false positive (FP), and true negative (TN) as presented in the 2×2 contingency sub-table in Table 1. From these four basic measurements, all the other relevant statistical measures can then be derived.

TABLE 1
Summary of statistical performance measures and their definitions used in reporting test results.

| Test | Reference Classification | | | Performance |
|---|---|---|---|---|
| Classification | Positive | Negative | Total | Measures |
| Positive | True Positive | False Positive | All Positive Test Cases | Positive Posttest Odds |
| | TP | FP | TP + FP | TP/FP |
| | | | | Positive Predictive Value (PPV) |
| | | | | TP/(TP + FP) |
| Negative | False Negative | True Negative | All Negative Test Cases | Negative Posttest Odds |
| | FN | TN | FN + TN | TN/FN |
| | | | | Negative Predictive Value |
| | | | | (NPV) |
| | | | | TN/(FN + TN) |
| Total | All Positive Cases | All Negative | All Cases | Positive Pretest Odds |
| | TP + FN | Cases | TP + FN + FP + TN | (TP + FN)/(FP + TN) |
| | | FP + TN | Prevalence | Negative Pretest Odds |
| | | | (TP + FN)/ | (FP + TN)/(TP + FN) |
| | | | (TP + FN + FP + TN) | |
| Performance | Sensitivity (Se) | False Positive | Positive Like- | Overall Accuracy |
| Measures | TP/(TP + FN) | Rate = 1 − | lihood Ratio | (TP + TN)/ |
| | | Specificity | (LR+) | (TP + FN + FP + TN) |
| | | FP/(FP + TN) | Sensitivity/(1 − | |
| | | | Specificity) | |
| | False Negative | Specificity (Sp) | Negative Like- | Diagnostic odds ratio |
| | Rate = 1 − | TN/(FP + | lihood Ratio | (LR+)/(LR−) |
| | Sensitivity | TN) | (LR−) | (Se × Sp)/(1 − Se) × (1 − Sp) |
| | FN/(TP + FN) | | (1 − Sensitivity)/ | (TP/FP) × (TN/FN) |
| | | | Specificity | |

Sensitivity (Se) indicates the ability of a test to identify positive cases; a test with high sensitivity has few false negatives (e.g., incorrectly identifies a patient as not having a disease). Specificity (Sp) indicates the ability of a test to identify negative cases; a test with high specificity has few false positives (e.g., incorrectly identifies a patient as having a disease). Positive predictive value (PPV) provides the probability of being true positive when the test is positive. Negative predictive value (NPV) provides the probability of being true negative when the test is negative.

Positive likelihood ratio (LR+) and negative likelihood ratio (LR−), which combine both the sensitivity and specificity of the test, provide estimates of how much the result of a test will change the odds of being positive and negative, respectively. Finally, overall accuracy (ACC) is a single-valued performance measure calculated as the ratio of all the correct classification (both true positive and true negative) to the total test cases.

For a complete description of the performance of a test, both sensitivity and specificity need to be reported. In addition, for most applications, both positive predictive value and negative predictive value and/or positive posttest odds and negative posttest odds are often included as part of the test performance reporting. Because once a test is positive, one is interested to know the predictive value of the test, namely, the likelihood (or probability) that the positive test is indeed a positive case. In addition, overall accuracy is often used to report the performance results because it is a single-valued performance measure that incorporates both sensitivity and specificity.

Prevalence-Dependent Performance Measures

Unlike sensitivity and specificity, which are independent of the prevalence of the condition being tested, the other performance measures PPV, Positive Posttest Odds, NPV, Negative Posttest Odds, and ACC depend on the test prevalence. Because of this prevalence-dependent nature of these measures, it is very important to understand the impact of test prevalence when using these performance measures in reporting and interpreting the test results. In the following, these prevalence-dependent performance measurements are discussed in detail.

Positive and Negative Predictive Values

Given the performance of a test as specified by the sensitivity and specificity, PPV and NPV as a function of the prevalence can be calculated using the following equations:

$$P P V = \frac{T P}{T P + F P} = \frac{T P / N}{T P / N + F P / N} \qquad ( 1 )$$

### Where:

N=TP+FN+FP+TN (2)

Substitute TP/N and FP/N with the following two equations, respectively:

$$\begin{matrix}\frac{T P}{N} = \frac{T P}{T P + F N} \times \frac{T P + F N}{N} = Se \times Prevalence & ( 3 ) \\ \frac{F P}{N} = \frac{F P}{F P + T N} \times \frac{F P + T N}{N} = ( 1 - S p ) \times ( 1 - Prevalence ) & ( 4 )\end{matrix}$$

PPV can then be expressed in terms of Se, Sp, and Prevalence in the following equation:

$$P P V = \frac{Se \times Prevalence}{Se \times Prevalence + ( 1 - S p ) \times ( 1 - Prevalence )} \qquad ( 5 )$$

Using Eqs. (3) and (4), positive posttest odds can also be expressed in terms of Se, Sp, and prevalence as shown below:

$$Positive Posttest Odds = \frac{T P}{F P} = \frac{T P / N}{F P / N} = \frac{Se \times Prevalence}{( 1 - S p ) \times ( 1 - Prevalence )} \qquad ( 6 )$$

From Eq. (6), positive posttest odds can also be expressed in terms of positive likelihood ratio (LR+) and positive pretest odds:

$$Positive Posttest Odds = \frac{Se}{1 - Sp} \times \frac{Se \times Prevalence}{1 - Prevalence} = ( {LR}_{+} ) \times Positive Posttest Odds \qquad ( 7 )$$

Similarly, NPV and negative posttest odds can also be calculated in terms of Se, Sp, and prevalence as shown in the following equations:

$$\begin{matrix} NPV = \frac{T N}{F N + T N} = \frac{T N / N}{F N / N + T N / N} & ( 8 ) \\ \frac{T N}{N} = \frac{T N}{F P + T N} \times \frac{F P + T N}{N} = Sp \times ( 1 - Prevalence ) & ( 9 ) \\ \frac{F N}{N} = \frac{F N}{T P + F N} \times \frac{T P + F N}{N} = ( 1 - Se ) \times Prevalence & ( 10 ) \\ NPV = \frac{S p \times ( 1 - Prevalence )}{( 1 - S e ) \times Prevalence + Sp \times ( 1 - Prevalence )} & ( 11 ) \\ Negative Posttest Odds = \frac{T N}{F N} = \frac{T N / N}{F N / N} = \frac{Sp \times ( 1 - Prevalence )}{( 1 - S e ) \times Prevalence} & ( 12 ) \\ Negative Posttes Odds = \frac{S p}{1 - S e} \times \frac{1 - Prevalence}{Prevalence} = ( L {R}_{-} ) \times Negative Pretest Odds & ( 13 )\end{matrix}$$

As an example to show the relationship of prevalence and PPV, three separate tests, each with 10,000 test cases but different prevalence levels, are conducted. The TP, FN, FP and TN numbers for all three tests are shown in Table 2. From these numbers, the sensitivity, specificity, prevalence, and PPV can be calculated directly using the definitions provided in Table 1 and the results are summarized in Table 3. The results show that while all three tests have the same sensitivity (95%) and specificity (95%), the PPVs for these three tests are very different. The PPVs are 95%, 68%, and 16% for prevalence levels of 50%, 10%, and 1%, respectively. The PPV depends on the pretest prevalence. A low prevalence yields a low PPV. The first test with a prevalence level of 50% produces a high PPV of 95%. On the other hand the third test with a prevalence of 1% produces a very low PPV value of only 16%, which is not a very useful test.

TABLE 2
Summary of test results for an example of understanding
the relationship between positive predictive value and
prevalence. N+ = Positive cases, N− = Negative cases

| | Test #1 | | Test #2 | | | |
|---|---|---|---|---|---|---|
| | (10,000 Cases; | | (10,000 Cases; | | Test #3 | |
| | Prevalence = | | Prevalence = | | (10,000 Cases; | |
| | 50%) | | 10%) | | Prevalence = 1%) | |
| Test | N+ | N− | N+ | N− | N+ | N− |
| Results | (5,000) | (5,000) | (1,000) | (9,000) | (100) | (9,900) |
| Test | 4,750 | 250 | 950 | 450 | 95 | 495 |
| positive | | | | | | |
| Test | 250 | 4,750 | 50 | 8,550 | 5 | 9,405 |
| negative | | | | | | |

TABLE 3
Summary of test performance calculated
from the results provided in Table 2

| | Test | Test | Test |
|---|---|---|---|
| Performance Results | #1 | #2 | #3 |
| Sensitivity | 95% | 95% | 95% |
| Specificity | 95% | 95% | 95% |
| Prevalence | 50% | 10% | 1% |
| Positive Predictive | 95% | 68% | 16% |
| Value | | | |

Note also that if the test sensitivity and specificity are known for a diagnostic test, the relationship of the prevalence and PPV as shown in Table 3 can also be obtained using Eq. (5). Although the PPV and NPV can be determined easily using the described equations, the relationships of these performance measures to the prevalence levels are not obvious from the equations.

Because the prevalence can significantly impact the test performance results it is very important to know the prevalence in order to interpreting the test results. While for many applications, one can potentially achieve a higher PPV by selecting only cases to be tested with high pretest likelihood (prevalence). However, for some applications, for example in real-time ECG/arrhythmia monitoring this may not be an option, since very often all patients are monitored regardless whether they are likely to have arrhythmia or not.

Overall Accuracy

Overall accuracy (ACC), defined as the ratio of all correct classification (true positive and true negative) to the total test cases (see Table 1), can be expressed in terms of sensitivity, specificity, and prevalence from the following equation:

$$A C C = \frac{T P + T N}{N} = \frac{T P}{N} + \frac{T N}{N} \qquad ( 14 )$$

Substitute TP/N and TN/N with the following two equations, respectively:

$$\begin{matrix}\frac{T P}{N} = \frac{T P}{T P + F N} \times \frac{T P + F N}{N} = Se \times Prevalence & ( 15 ) \\ \frac{T N}{N} = \frac{T N}{F P + T N} \times \frac{F P + T N}{N} = Sp \times ( 1 - Prevalence ) & ( 16 )\end{matrix}$$

ACC can then be expressed in terms of Se, Sp, and prevalence in the following equation:

ACC=Se×Prevalence+Sp×(1−Prevalence) (17)

This equation shows that ACC is a weighted sum of the sensitivity and specificity of the test. The weighting factor for the sensitivity is the prevalence and the weighting factor for the specificity is the complement of the prevalence. Thus, for an algorithm with performance specified by sensitivity and specificity, ACC will vary depending on the prevalence of the positive cases in the test.

To illustrate this relationship, Table 4 shows the ACC values for three different algorithms (Algorithm A: Se=60%, Sp=40%, Algorithm B: Se=40%, Sp=60%, and Algorithm C: Se=Sp=60%) tested at three different levels of prevalence (90%, 50%, and 10%). When the prevalence is greater than 50% (Table 4, Prevalence=90%), ACC will be higher for an algorithm with higher sensitivity than specificity (Algorithm A). On the other hand, when the prevalence is less than 50% (Table 4, Prevalence=10%), ACC will be higher for an algorithm with higher specificity than sensitivity (Algorithm B). When sensitivity and specificity are the same (Algorithm C), regardless of the prevalence level, ACC will be the same as the sensitivity and specificity. When the prevalence level is 50%, ACC will equal to the mathematical average of the sensitivity and specificity. Note also for an algorithm with Se>Sp (Algorithm A), ACC decreases as prevalence drops. On the other hand, for an algorithm with Sp>Se (Algorithm B), ACC increases as prevalence decrease.

TABLE 4
Overall accuracy (ACC) as a function of the prevalence level.

| | Overall Accuracy | | |
|---|---|---|---|
| | Algorithm A | Algorithm B | Algorithm C |
| | Se = 60% | Se = 40% | Se = 60% |
| Prevalence | Sp = 40% | Sp = 60% | Sp = 60% |
| 90% | 58% | 42% | 60% |
| 50% | 50% | 50% | 60% |
| 10% | 42% | 58% | 60% |

Accuracy Paradox

Because of this prevalence-dependent nature of the overall accuracy, it has created the so-called “accuracy paradox”, where a useless test may have a higher overall accuracy than a more useful test with a lower value of overall accuracy. The accuracy paradox is illustrated in the example as shown in Table 5.

TABLE 5
Summary of test performance results for two
different classification algorithms A and B.

| Algorithms | | D+ | D− | Prevalence |
|---|---|---|---|---|
| Tested | | (500) | (9,500) | (5%) |
| A | Test+ | 450 | 950 | PPV = 32% |
| | Test− | 50 | 8,550 | |
| | Results | Se = 90% | Sp = 90% | Acc = 90% |
| B | Test+ | 0 | 0 | PPV = N/A |
| | Test− | 500 | 9,500 | |
| | Results | Se = 0% | Sp = 100% | Acc = 95% |

In Table 5, algorithms A and B are tested using a dataset with 10,000 cases and 5% prevalence. Algorithm A has Se, Sp, and PPV equal to 90%, 90%, and 32% respectively. Algorithm B, on the other hand, is a totally useless algorithm. The algorithm calls all cases negative (Sp=100%) and fails to detect any positive cases (Se=0%). However, algorithm B has a higher ACC (95%) than algorithm A (90%).

Although, it is intuitively reasonable to assume that the overall accuracy should be a very useful single-valued performance measure, this example clearly shows that in some cases the overall accuracy is not a reliable performance measure.

It is also important to know that while database used in testing of detection and classification algorithms must contain sufficient number of positive cases (thus, high prevalence) in order to accurately measure the sensitivity of the algorithm being evaluated, the performance results thus obtained may not be relevant in actual application since the prevalence of the targeted application for the test may have a very different prevalence level (usually much lower). Thus, it is very important that the posttest likelihood needs to be reported using the actual prevalence level of the application that the test is targeted for.

The described performance measures used in reporting the performance results, as summarized in Table 1, have been used for decades in many applications. There are several problems with the use of these performance measures for reporting the performance results. First, currently there are no efficient ways to present and summarize the large number of the described performance measures. As such, very often only a subset of these performance measures is included in the reporting of the performance results. Secondly, the complex relationships of these performance measures as discussed previously are often not easy to understand and interpret with just the equations. Thirdly, the most important equations used for calculating PPV, NPV, positive posttest odds, negative posttest odds, and ACC from Se, Sp, and prevalence are very difficult to memorize and thus making assessment of the results using different values of Se, Sp, and prevalence difficult.

In order to better present the performance results of a diagnostic test, embodiments of a new graphical presentation for reporting the performance results of a diagnostic test is disclosed herein. In this two dimensional rectangular graph the left vertical axis is sensitivity, the right vertical axis is specificity, and the horizontal axis is prevalence. The graph has a 2×2 areas defined by three straight lines: a vertical prevalence line, a horizontal sensitivity line, and a horizontal specificity line. These four areas, marked as TPn, FNn, FPn, and TNn, are the normalized values of true positive, false negative, false positive, and true negative, respectively. Other performance measures are represented as line segments (e.g., sensitivity, specificity, and prevalence), ratio of line segments (e.g., positive and negative likelihood ratios), ratio of areas (e.g., positive and negative predictive values), and sum of areas (e.g., overall accuracy). Using this graph a total of 19 performance measures may be presented and visualized simultaneously. With the ability to directly visualize these performance measures in a single graph, it allows the complex relationships of all performance measures to be understood and interpreted more easily by users evaluating the performance measures.

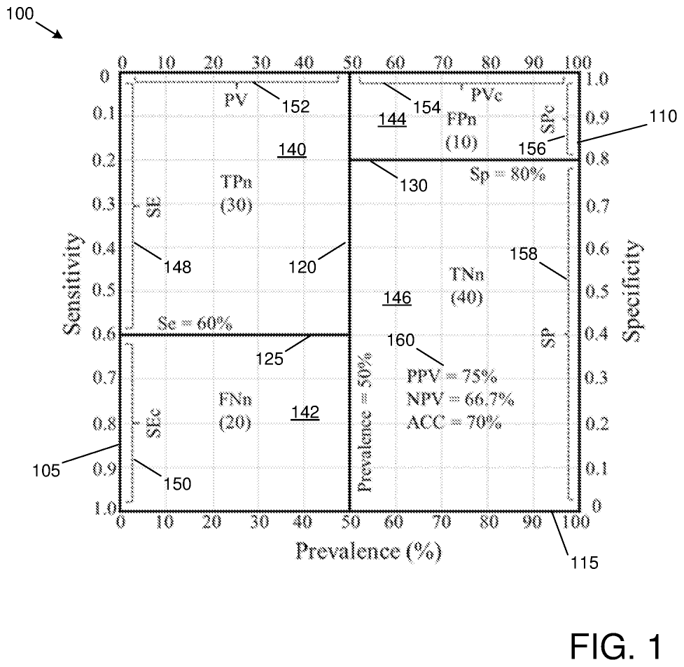

FIG. 1 illustrates an example of the graphical presentation for performance reporting. The graphical performance display 100 includes a two dimensional rectangular graph such that the left vertical axis 105 is sensitivity (0 to 1 values from top to bottom), the right vertical axis 110 is specificity (0 to 1 values from bottom to top), and the horizontal axis 115 is prevalence (0 to 100° A left to right). The graph has 2×2 areas defined by three straight lines: a vertical prevalence line 120, a horizontal sensitivity line 125, and a horizontal specificity line 130. There are four areas, marked as TPn 140, FNn 142, FPn 144, and TNn 146 that are the normalized values of TP, FN, FP, and TN, respectively. Other performance measures are represented as line segments (e.g., Se 148, Sp 158, and Prevalence 152), ratio of line segments (e.g., LR+, LR−), ratio of areas (e.g., Positive posttest odds, Negative posttest odds, PPV, NPV), and sum of areas (e.g., ACC). Using this graphical performance display 100 a total of 19 performance measures can be presented and visualized simultaneously. With the ability to directly visualize these performance measures, it allows the complex relationships of all performance measures to be understood and interpreted more easily.

In addition to the elements described above, the numerical values for PPV, NPV, and ACC 160 are also displayed as shown in FIG. 1. Additional numerical values for LR+, LR−, positive pretest odds, negative pretest odds, positive posttest odds, and negative posttest odds may also be included as part of 160 as desired.

The generation and use of the graphical performance display 100 will now be described as a process including obtaining the required input data and then constructing the performance graph.

Obtain the required input data

Step 1—Acquire the basic performance statistics from the test performed: TP, FN, FP, and TN as defined in Table 1.

Step 2—Calculate the values of the three performance measures Se, Sp, and Prevalence (using the equations described in Table 1): note: skip the above this step if Se, Sp, and Prevalence are already available.

Construct the Performance Graph

Step 1—Plot a rectangular box with scales on all four sides according to the specifications listed in the table below:

| Axis | Axis label | Scales |
|---|---|---|
| Left vertical | Sensitivity | From top to bottom: 0 to 1.0 in |
| | | increment of 0.1 |
| Right vertical | Specificity | From bottom to top: 0 to 1.0 in |
| | | increment of 0.1 |
| Bottom horizontal | Prevalence (%) | From left to right: 0 to 100 in |
| | | increment of 10 |
| Top horizontal | None | From left to right: 0 to 100 in |
| | | increment of 10 |

Note that the scales are defined such that the total area of the graphical performance display is 100. An alternative scale set that can also be used is to change the sensitivity and specificity to 0 to 100% in increment of 10% and the prevalence to 0 to 1.0 in increments of 0.1.

Step 2—Plot the vertical prevalence line 120 from top to bottom according to the prevalence value. In some embodiments, enter the prevalence value next to the prevalence line. On the prevalence axis 115, two line segments PV 152 and PVc 154 are created with values defined below:

PV=Prevalence line segment=prevalence×100 (18)

PV_{c}=Complement of prevalence line segment=(1−Prevalence)×100 (19)

These two line segments define the positive pretest odds and the negative pretest odds:

$$\begin{matrix}Positive pretest odds = \frac{Prevalence}{1 - Prevalence} = \frac{P V}{P {V}_{c}} & ( 20 ) \\ Negative pretest odds = \frac{1 - Prevalence}{Prevalence} = \frac{{PV}_{c}}{PV} & ( 21 )\end{matrix}$$

Step 3—Plot the horizontal sensitivity line 125 from the left vertical axis to the vertical prevalence line according to the sensitivity value. In some embodiments, display the sensitivity value next to the sensitivity line. On the sensitivity axis, two line segments SE 148 and SEc 150 are created with values defined below:

SE=Sensitivity line segment=Se (22)

SE_{c}=Complement of sensitivity line segment=1−Se (23)

Step 4—Plot the horizontal specificity line 130 from the right vertical axis to the vertical prevalence line according to the specificity value. In some embodiments, display the specificity value next to the specificity line. On the specificity axis, two lines segments SP 158 and SPc 156 are created with values defined below:

SP=Specificity line segment=Sp (24)

SP_{c}=Complement of specificity line segment=1−Sp (25)

The four sensitivity and specificity line segments define LR+ and LR− as shown below:

$$\begin{matrix}Positive likelihood ratio ( LR + ) = \frac{S e}{1 - S p} = \frac{S E}{S {P}_{c}} & ( 26 ) \\ Negative likelihood ratio ( L R - ) = \frac{1 - S e}{S p} = \frac{S {E}_{c}}{S P} & ( 27 )\end{matrix}$$

Step 5—Calculate the four areas defined by the three performance lines and the six line segments as defined above using the following equations:

UL (Upper left area)=SE×PV=Se×Prevalence×100 (28)

UR (Upper right area)=SP_{c}×PV_{c}=(1−Sp)×(1−Prevalence)×100 (29)

LL (Lower left area)=SE_{c}×PV=(1−Se)×Prevalence×100 (30)

LR (Lower right area)=SP×PV_{c}=Sp×(1−Prevalence)×100 (31)

Equations (28)-(31) may be expressed in terms of the four basic performance statistics TP, FN, FP, and TN as shown below:

$$\begin{matrix}\begin{matrix}UL ( Upper left area ) = Se \times Prevalence \times 100 \\ = \frac{TP}{TP + FN} \times \frac{TP + FN}{N} \times 100 \\ = \frac{TP}{N} \times 100 = {TP}_{n}\end{matrix} & ( 32 ) \\ \begin{matrix}UR ( Upper right area ) = ( 1 - Sp ) \times ( 1 - Prevalence ) \times 100 \\ = \frac{FP}{FP + TN} \times \frac{FP + TN}{N} \times 100 \\ = \frac{FP}{N} \times 100 = {FP}_{n}\end{matrix} & ( 33 ) \\ \begin{matrix}LL ( Lower left area ) = ( 1 - Se ) \times Prevalence \times 100 \\ = \frac{FN}{TP + FN} \times \frac{TP + FN}{N} \times 100 \\ = \frac{FN}{N} \times 100 = {FN}_{n}\end{matrix} & ( 34 ) \\ \begin{matrix}LR ( Lower right area ) = Sp \times ( 1 - Prevalence ) \times 100 \\ = \frac{TN}{FP + TN} \times \frac{FP + TN}{N} \times 100 \\ = \frac{TN}{N} \times 100 = {TN}_{n}\end{matrix} & ( 35 )\end{matrix}$$

Where TPₙ 140, FNₙ 142, FPₙ 144, and TNₙ 146 are defined as below:

$$\begin{matrix}T {P}_{n} = \frac{T P}{N} \times 100 = Normalized True Positive & ( 36 ) \\ F {P}_{n} = \frac{F P}{N} \times 100 = Normalized False Positive & ( 37 ) \\ F {N}_{n} = \frac{F N}{N} \times 100 = Normalized False Negative & ( 38 ) \\ T {N}_{n} = \frac{T N}{N} \times 100 = Normalized True Negative & ( 39 )\end{matrix}$$

The range of these normalized values is from 0 to 100 and the sum of them is equal to 100 as indicated below:

$$\begin{matrix} 0 \leq T {P}_{n} , {FP}_{n} , {FN}_{n} , {TN}_{n} \leq 100 & ( 40 ) \\ T {P}_{n} + F {P}_{n} + F {N}_{n} + T {N}_{n} = \frac{T P + F P + F N + T N}{N} \times 100 = 100 & ( 41 )\end{matrix}$$

Step 6—Label the upper-left, lower-left, upper-right, and lower-right cells as TPₙ 140, FNₙ 142, FPₙ 144, and TNₙ 146 respectively, and enter the corresponding values calculated from Equations (36)-(39).

Step 7—Calculate PPV, NPV, and ACC 160 from the equations as shown below:

$$\begin{matrix}\begin{matrix}PPV = \frac{T P}{T P + F P} = \frac{( T P / N ) \times 100}{( T P / N ) \times 100 + ( F P / N ) \times 100} \\ = \frac{T {P}_{n}}{T {P}_{n} + F {P}_{n}} = \frac{U L}{U L + U R}\end{matrix} & ( 42 ) \\ \begin{matrix}NPV = \frac{T N}{F N + T N} = \frac{( T N / N ) \times 100}{( F N / N ) \times 100 + ( T N / N ) \times 100} \\ = \frac{T {N}_{n}}{F {N}_{n} + T {N}_{n}} = \frac{L R}{L L + L R}\end{matrix} & ( 43 ) \\ \begin{matrix}ACC = \frac{T P + T N}{N} = \frac{T P}{N} + \frac{T N}{N} \\ = ( \frac{T P}{N} \times 100 + \frac{T N}{N} \times 100 ) / 100 \\ = ( T {P}_{n} + T {N}_{n} ) / 100 = ( U L + L R ) / 1 \; 00\end{matrix} & ( 44 )\end{matrix}$$

Note that from Equations (5), (11), and (17), PPV, NPV, and ACC can also be obtained directly using the area equations (13)-(16) as shown below:

$$\begin{matrix}\begin{matrix}PPV = \frac{Se \times Prevalence}{Se \times Prevalence + ( 1 - Sp ) \times ( 1 - Prevalence )} \\ = \frac{UL / 100}{UL / 100 + UR / 100} \\ = \frac{UL}{UL + UR}\end{matrix} & ( 45 ) \\ \begin{matrix}NPV = \frac{Sp \times ( 1 - Prevalence )}{( 1 - Se ) \times Prevalence + Sp \times ( 1 - Prevalence )} \\ = \frac{LR / 100}{LL / 100 + LR / 100} \\ = \frac{LR}{LL + LR}\end{matrix} & ( 46 ) \\ \begin{matrix}ACC = Se \times Prevalence + Sp \times ( 1 - Prevalence ) \\ = \frac{UL}{100} + \frac{LR}{100} \\ = ( UL + LR ) / 100\end{matrix} & ( 47 )\end{matrix}$$

Step 8—Display the values of PPV, NPV, and ACC 160 calculated from Equations (45)-(47). In another embodiment some or all of the values LR+, LR−, positive pretest odds, negative pretest odds, positive posttest odds, and negative posttest odds may also be displayed along with PPV, NPV, and ACC.

In summary, all commonly used performance measures as listed in Table 1 are graphically shown in FIG. 1 as summarized below:
1) TPn 140 (Normalized TP)=Upper-left area
2) FPn 144 (Normalized FP)=Upper-right area;
3) FNn 142 (Normalized FN)=Lower-left area;
4) TNn 146 (Normalized TN)=Lower-right area
5) Sensitivity (Se) 148=Line segment SE
6) False negative rate 150=1−Se=Line segment SEc
7) Specificity (Sp) 158=Line segment SP
8) False positive rate 156=1−Sp=Line segment SPc
9) Positive likelihood ratio (LR+)=Ratio of line segments SE to SPc
10) Negative likelihood ratio (LR−)=Ratio of line segments SEc to SP
11) Prevalence 152=Line segment PV
12) 1−Prevalence 154=Line segment PVc
13) Positive pretest odds=Ratio of line segments PV to PVc
14) Negative pretest odds=Ratio of line segments PVc to PV
15) Positive posttest odds=Ratio of upper-left area to upper-right area
16) Positive predictive value (PPV)=Ratio of upper-left area to the sum of upper-left and upper-right areas
17) Negative posttest odds=Ratio of lower-right area to lower-left area
18) Negative predictive value (NPV)=Ratio of lower-right area to the sum of lower-left and lower-right areas
19) Overall accuracy (ACC)=Sum of upper-left and lower-right areas

Several examples are shown below to illustrate the usefulness of the graphical performance plot as described in FIG. 1.

### EXAMPLE 1

### Relationship Between PPV and Prevalence

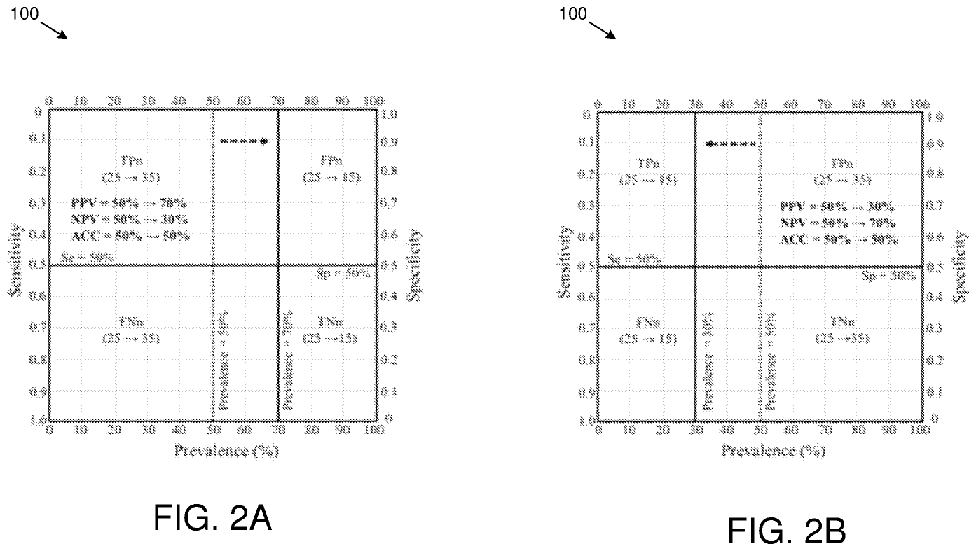

Although PPV and NPV can be calculated from Eqs. (5) and (11), respectively, their relationships to Se, Sp, and prevalence are not obvious. With the aid of the graphical performance display, it becomes relatively easy to demonstrate the relationship. FIGS. 2A and 2B illustrate how the graphical performance display is used to understand how changing the prevalence value affects various other performance measures. In these examples, both Se and Sp remain unchanged at 50%. Graphically, PPV is the ratio of the upper-left area to the sum of the upper-left and upper-right areas. Thus PPV equals 50% when prevalence is at 50% level because the upper-left and upper-right areas are the same. However, as shown in FIG. 2A, PPV will increase from 50% to 70% when prevalence is increased because now the upper-left area becomes larger than the upper-right area. On the other hand, as shown in FIG. 2B, PPV will decrease from 50% to 30% when the prevalence is decreased because now the upper-left area becomes smaller than the upper-right area. These relationships remain the same whether Se>Sp or Sp>Se, because moving the vertical prevalence line only changes the width but not the height of the two areas used in determining the results. Thus, using the graphical performance graph, it is very easy to show that increasing prevalence will always improve PPV regardless of the values of Se and Sp.

### EXAMPLE 2

### How to Improve PPV?

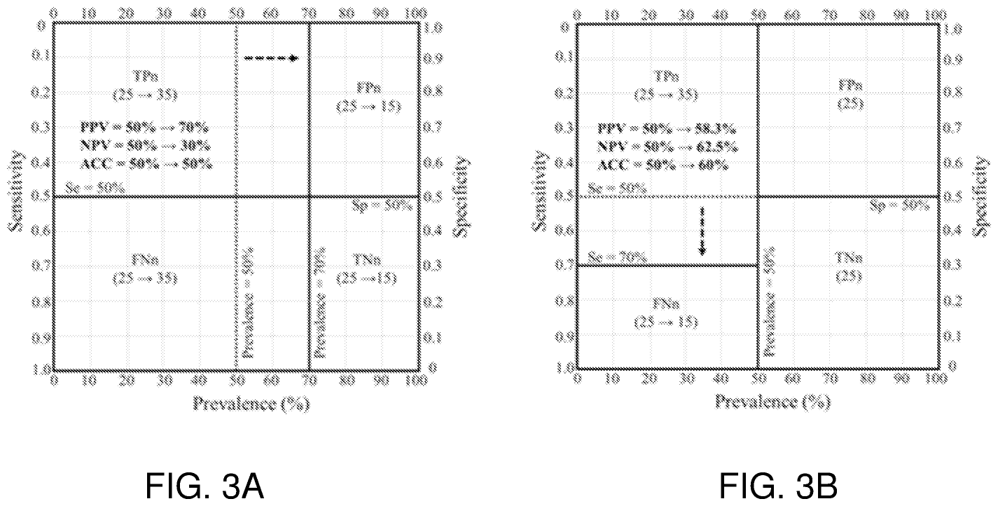

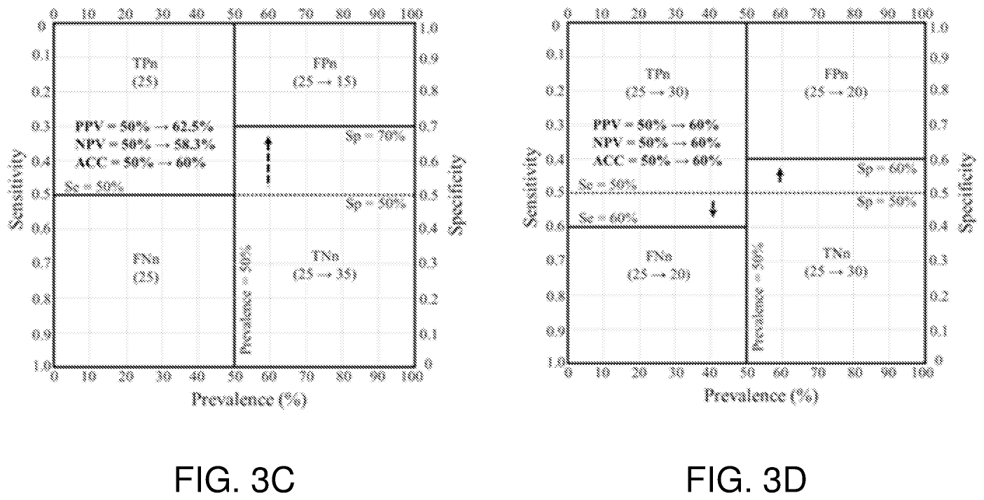

FIGS. 3A-3D illustrate that three lines may be moved to increase TP (upper-left) area and/or decrease FP (upper-right) area to improve the PPV. Which strategy is the best given a possible change of 20% in total? The performance results are shown in Table 6 for the following four strategies: 1) increase the upper-left area and decrease the upper-right area by increasing prevalence by 20% (FIG. 3A); 2) increase the upper-left area by increasing the sensitivity by 20% (FIG. 3B); 3) decrease the upper-right area by increasing the specificity by 20% (FIG. 3C); and 4) increase the upper-left area and decrease the upper-right area by increasing both the sensitivity and specificity by 10% (FIG. 3D). The positive likelihood ratio LR+, computed as the ratio of the SE line segment to the SPc line segment, Se/(1−Sp), is also shown in the table. The higher the LR+ value the more useful the algorithm is. When the value is 1 the diagnostic test does not alter the positive posttest value from the positive pretest value as shown in the following equation:

$$\begin{matrix}\begin{matrix}PPV = \frac{Se \times Prevalence}{Se \times Prevalence + ( 1 - Sp ) \times ( 1 - Prevalence )} \\ = \frac{[ Se / ( 1 - Sp ) ] \times Prevalence}{[ Se / ( 1 - Sp ) ] \times Prevalence + ( 1 - Prevalence )}\end{matrix} \\ When Se / ( 1 - Sp ) = 1 , & ( 48 ) \\ PPV = \frac{Prevalence}{Prevalence + ( 1 - Prevalence )} = Prevalence & ( 49 )\end{matrix}$$

TABLE 6
Summary on how to improve PPV based on results from FIG. 3

| | LR+ | PPV (%) | NPV (%) | ACC (%) |
|---|---|---|---|---|
| Baseline: Se, Sp, | 1.0 | 50 | 50 | 50 |
| Prevalence = 50% | | | | |
| Increase prevalence by 20% | 1.0 | 70 | 30 | 50 |
| Increase Sp by 20% | 1.67 | 62.5 | 58.3 | 60 |
| Increase both Se and Sp by | 1.50 | 60 | 60 | 60 |
| 10% | | | | |
| Increase Se by 20% | 1.40 | 58.3 | 62.5 | 60 |

Instead of improving the performance of the diagnostic algorithm, strategy #1 (FIG. 3A) improves PPV performance by selecting test subjects with higher pretest likelihood. Very often this is the most effective strategy in improving PPV in many applications since the width of both upper left and upper right areas are increased and decreased simultaneously for the same amount.

Note also that in this case LR+=1, as such the diagnostic test itself does not improve PPV, rather the improved PPV is a direct result of the higher pretest likelihood (prevalence). Thus, both have the same value of 70% as seen from Eq. (49). For the other three strategies, as expected, higher LR+ value will result in higher PPV. The best strategy is to increase Sp by 20%. Note also that the ACC values are the same for the three described strategies because the sum of the upper-left and lower right areas remains the same regardless how the 20% performance gain is allocated.

### EXAMPLE 3

### ACC Behaviors

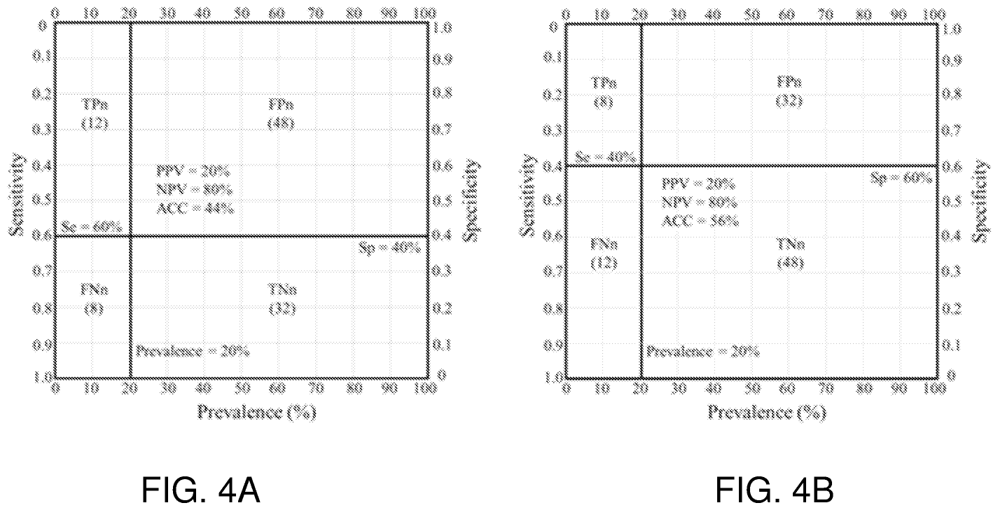

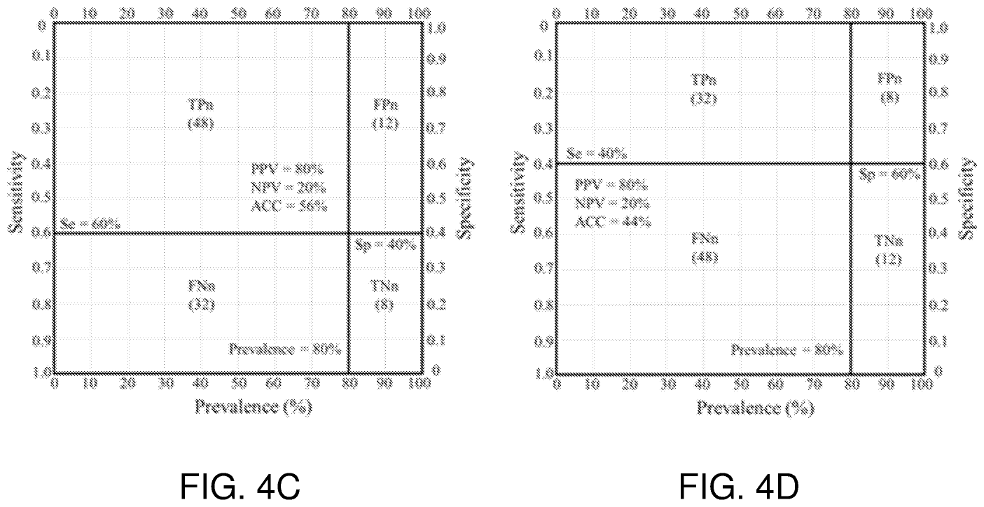

FIGS. 4A-4D illustrate ACC behavior for increased prevalence level for Se>Sp vs. Se<Sp. Previously in Example 1, it was shown that PPV can be improved by increasing the prevalence value regardless whether Se>Sp or Sp>Se. However, for ACC the behavior is different. When Se (60%)>Sp (40%) (FIGS. 4A and 4C), the amount of increase of the upper left area (TPn) is greater than the amount of decrease of the lower right area (TNn), thus resulting in a higher ACC value from 44% to 56% with an increase of prevalence from 20% to 80%. On the other hand, when Se (40%)<Sp (60%) (FIGS. 4B and 4D), the amount of increase on the upper left area (TPn) is less than the amount of decrease of the lower right area (TNn), thus resulting in a lower ACC value from 56% to 44% with an increase of prevalence from 20% to 80%.

### EXAMPLE 4

### ACC Paradox

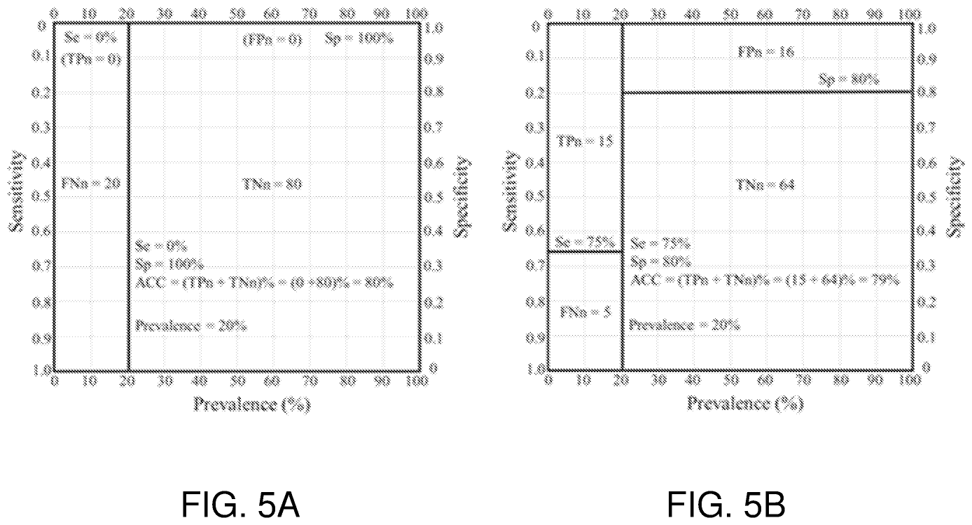

FIGS. 5A and 5B illustrate the ACC paradox. FIG. 5A illustrates a diagnostic algorithm with Se=0% and Sp=100° A, and thus a rather useless diagnostic algorithm because it will not be able to detect any positive cases. With a prevalence level of 20%, it has an ACC value of 80%. FIG. 5B illustrates another algorithm with Se=75% and Sp=80% and thus a much more useful diagnostic algorithm. However, for this algorithm the ACC value is 79% which is actually less than the algorithm shown in FIG. 5A. From the figure it is clear that the ACC paradox occurs whenever the TNn area in FIG. 5A is greater than the sum of the TPn and TNn areas. This example demonstrate the so-called “accuracy paradox” where a totally useless test may have a higher ACC value than a more useful test with a lower ACC value.

A new graphical performance display is described herein. This graphical performance display summarizes all the commonly used performance measures in a single graph. The ability of the graphical performance display in providing an extremely simple graph that allows for direct visualization of the relationships of all the performance measures is finally making the complex relationships easy to follow and understand. The graphical performance display is not product specific; it can be used in performance reporting for any diagnostic or detection algorithms.

The graphical performance display may be used in many applications. The graphical performance display may be used for performance reporting for all diagnostic, detection, or classification algorithms. The graphical performance display may also be used as an excellent teaching tool in describing and explaining all the performance measures used in performance reporting. The graphical performance display may be adopted widely in teaching the subject of performance measures. The graphical performance display may be incorporated into textbooks or other printed publication on the subject of diagnostic test performance. The graphical performance display may be adopted as a standard for performance results reporting.

In addition, the graphical performance display may be implemented as an interactive application for performance results analysis and presentation. In such an application, that data may be presented on a display to a user, and the user may interactively adjust different values in the graphical performance display, such as for example, prevalence, specificity, sensitivity, etc. The user can then quickly and easily see how these change affect various other performance measures, as the changes in values are shown to the user. Such changes may be implemented by an interface where the user types in new values to be used in the graphical performance display. In another embodiment, a user interface may allow the user to select and drag or move the prevalence line 120, the sensitivity line 125, or the specificity line 130 to see the effects on other performance measures. The interface may also be configured to keep a history of the changes made by the user, that may be reviewed and displayed again by the user. Such an interactive graphical performance display allows a user to gain greater insights into the data results and how seeking to vary certain results will affect other performance measures. Further, tables of data such as some of those shown above may also be generated and presented on the display to help the user gain insight into the performance data. The user interface may be configurable to allow the user to determine which specific data is shown including PPV, NPV, AAC, LR+, LR−, positive pretest odds, negative pretest odds, positive posttest odds, negative posttest odds, and tabulated data.

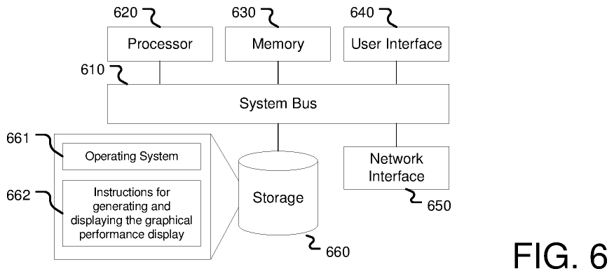

FIG. 6 illustrates an exemplary hardware diagram 600 for generating and displaying the graphical performance display illustrated in FIGS. 1-5. As shown, the device 600 includes a processor 620, memory 630, user interface 640, network interface 650, and storage 660 interconnected via one or more system buses 610. It will be understood that FIG. 6 constitutes, in some respects, an abstraction and that the actual organization of the components of the device 600 may be more complex than illustrated.

The processor 620 may be any hardware device capable of executing instructions stored in memory 630 or storage 660 or otherwise processing data. As such, the processor may include a microprocessor, a graphics processing unit (GPU), field programmable gate array (FPGA), application-specific integrated circuit (ASIC), any processor capable of parallel computing, or other similar devices.

The memory 630 may include various memories such as, for example L1, L2, or L3 cache or system memory. As such, the memory 630 may include static random-access memory (SRAM), dynamic RAM (DRAM), flash memory, read only memory (ROM), or other similar memory devices.

The user interface 640 may include one or more devices for enabling communication with a user and may present information to users. For example, the user interface 640 may include a display, a touch interface, a mouse, and/or a keyboard for receiving user commands. In some embodiments, the user interface 640 may include a command line interface or graphical user interface that may be presented to a remote terminal via the network interface 650. The user interface 640 may be used to display the graphical performance display.

The network interface 650 may include one or more devices for enabling communication with other hardware devices. For example, the network interface 650 may include a network interface card (NIC) configured to communicate according to the Ethernet protocol or other communications protocols, including wireless protocols. Additionally, the network interface 650 may implement a TCP/IP stack for communication according to the TCP/IP protocols. Various alternative or additional hardware or configurations for the network interface 650 will be apparent.

The storage 660 may include one or more machine-readable storage media such as read-only memory (ROM), random-access memory (RAM), magnetic disk storage media, optical storage media, flash-memory devices, or similar storage media. In various embodiments, the storage 660 may store instructions for execution by the processor 620 or data upon with the processor 620 may operate. For example, the storage 660 may store a base operating system 661 for controlling various basic operations of the hardware 600. The storage 662 may store instructions for generating and displaying the graphical performance display as described above.

It will be apparent that various information described as stored in the storage 660 may be additionally or alternatively stored in the memory 630. In this respect, the memory 630 may also be considered to constitute a “storage device” and the storage 660 may be considered a “memory.” Various other arrangements will be apparent. Further, the memory 630 and storage 660 may both be considered to be “non-transitory machine-readable media.” As used herein, the term “non-transitory” will be understood to exclude transitory signals but to include all forms of storage, including both volatile and non-volatile memories.

While the system 600 is shown as including one of each described component, the various components may be duplicated in various embodiments. For example, the processor 620 may include multiple microprocessors that are configured to independently execute the methods described herein or are configured to perform steps or subroutines of the methods described herein such that the multiple processors cooperate to achieve the functionality described herein. Such plurality of processors may be of the same or different types. Further, where the device 600 is implemented in a cloud computing system, the various hardware components may belong to separate physical systems. For example, the processor 620 may include a first processor in a first server and a second processor in a second server.

The graphical performance display described herein provides a technological improvement over current performance data analysis. Such current methods of displaying data use numbers or graphs (.e.g., receiver operating characteristic curve), but do not provide much insight into the relationships between the different performance measures. The graphical performance display provides a single and concise presentation of performance measures using a variety of graphical elements. This graphical performance display allows a user to understand the relationships and dependencies between the various performance measures. Also a user may change various performance measures, for example, prevalence, sensitivity, and specificity, and quickly see how the other performance measures change, to thus provide deeper insight into the performance measures, and what changes may be desirable in the underly tests or processes behind the performance data.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for generating a graphical performance display, comprising:
receiving performance measure data;
generating a graphical rectangular plot, having a first side indicating a prevalence value, a second side indicating a sensitivity value, and third side indicating a specificity value, wherein the second and third sides are opposite one another and a fourth side is opposite the first side;
drawing a prevalence line perpendicular to the first side between the first side and the fourth side based upon a prevalence value in the performance measure data;
drawing a sensitivity line perpendicular to the second side between the second side and the prevalence line based upon a sensitivity value in the performance measure data; and
drawing a specificity line perpendicular to the third side between the third side and the prevalence line based upon a sensitivity value in the performance measure data.

2. The method of claim 1, further comprising displaying the value of at least one of positive predictive value (PPV), negative predictive value (NPV), overall accuracy (ACC), positive likelihood ratio (LR+), negative likelihood ratio (LR−), positive pretest odds, negative pretest odds, positive posttest odds, and negative posttest odds

3. The method of claim 2, further comprising calculating the displayed value based upon the received performance measure data.

4. The method of claim 1, further comprising:
displaying a normalized true positive value (TPn) in a first rectangular area bounded by the second side, fourth side, prevalence line, and sensitivity line, wherein the TPn value is related to the area of the first rectangular area;
displaying a normalized false negative value (FNn) in a second rectangular area bounded by the second side, first side, prevalence line, and sensitivity line, wherein the FNn value is related to the area of the second rectangular area;
displaying a normalized false positive value (FPn) in a third rectangular area bounded by the third side, fourth side, prevalence line, and specificity line, wherein the FPn value is related to the area of the third rectangular area; and
displaying a normalized true negative value (TNn) in a fourth rectangular area bounded by the third side, first side, prevalence line, and specificity line, wherein the TNn value is related to the area of the fourth rectangular area.

5. The method of claim 4, further comprising calculating values for predictive value (PPV), negative predictive value (NPV), overall accuracy (ACC) using either the TPn, FNn, FPn, and TNn values or the areas for the first rectangular area, second rectangular area, third rectangular area, and fourth rectangular area.

6. The method of claim 2, further comprising:
receiving user input indicating a change in one of the prevalence, sensitivity, and specificity;
displaying a change in the location of the prevalence line, sensitivity line, or the specificity line based upon the received user input;
recalculating the displayed value; and
displaying the recalculated value.

7. The method of claim 6, wherein the user input includes selecting one of the prevalence line, sensitivity line, and specificity line and dragging it to a new position.

8. The method of claim 6, wherein the user input includes inputting one of prevalence value, sensitivity value, and specificity value.

9. The method of claim 6, further comprising generating and displaying a table of performance measure data and recalculated performance measure data.

10. The method of claim 1, wherein a data scale for the second and third sides increase in opposite directions.

11. A non-transitory machine-readable storage medium encoded with instructions for generating a graphical performance display, comprising:
instructions for receiving performance measure data;
instructions for generating a graphical rectangular plot, having a first side indicating a prevalence value, a second side indicating a sensitivity value, and third side indicating a specificity value, wherein the second and third sides are opposite one another and a fourth side is opposite the first side;
instructions for drawing a prevalence line perpendicular to the first side between the first side and the fourth side based upon a prevalence value in the performance measure data;
instructions for drawing a sensitivity line perpendicular to the second side between the second side and the prevalence line based upon a sensitivity value in the performance measure data; and
instructions for drawing a specificity line perpendicular to the third side between the third side and the prevalence line based upon a sensitivity value in the performance measure data.

12. The non-transitory machine-readable storage medium of claim 11, further comprising instructions for displaying the value of at least one of positive predictive value (PPV), negative predictive value (NPV), overall accuracy (ACC), positive likelihood ratio (LR+), negative likelihood ratio (LR−), positive pretest odds, negative pretest odds, positive posttest odds, and negative posttest odds

13. The non-transitory machine-readable storage medium of claim 12, further comprising instructions for calculating the displayed value based upon the received performance measure data.

14. The non-transitory machine-readable storage medium of claim 11, further comprising:
instructions for displaying a normalized true positive value (TPn) in a first rectangular area bounded by the second side, fourth side, prevalence line, and sensitivity line, wherein the TPn value is related to the area of the first rectangular area;
instructions for displaying a normalized false negative value (FNn) in a second rectangular area bounded by the second side, first side, prevalence line, and sensitivity line, wherein the FNn value is related to the area of the second rectangular area;
instructions for displaying a normalized false positive value (FPn) in a third rectangular area bounded by the third side, fourth side, prevalence line, and specificity line, wherein the FPn value is related to the area of the third rectangular area; and
instructions for displaying a normalized true negative value (TNn) in a fourth rectangular area bounded by the third side, first side, prevalence line, and specificity line, wherein the TNn value is related to the area of the fourth rectangular area.

15. The non-transitory machine-readable storage medium of claim 14, further comprising instructions for calculating values for predictive value (PPV), negative predictive value (NPV), overall accuracy (ACC) using either the TPn, FNn, FPn, and TNn values or the areas for the first rectangular area, second rectangular area, third rectangular area, and fourth rectangular area.

16. The non-transitory machine-readable storage medium of claim 12, further comprising:
instructions for receiving user input indicating a change in one of the prevalence, sensitivity, and specificity;
instructions for displaying a change in the location of the prevalence line, sensitivity line, or the specificity line based upon the received user input;
instructions for recalculating the displayed value; and
instructions for displaying the recalculated value.

17. The non-transitory machine-readable storage medium of claim 16, wherein the user input includes selecting one of the prevalence line, sensitivity line, or specificity line and dragging it to a new position.

18. The non-transitory machine-readable storage medium of claim 16, wherein the user input includes inputting one of prevalence value, sensitivity value, or specificity value.

19. The non-transitory machine-readable storage medium of claim 16, further comprising instructions for generating and displaying a table of performance measure data and recalculated performance measure data.

20. The non-transitory machine-readable storage medium of claim 11, wherein a data scale for the second and third sides increase in opposite directions.
